(19) United States
(12) Patent Application Publication
(10) Pub. No.: US 2024/0298974 A1
(43) Pub. Date: Sep. 12, 2024

(54) PRO-ICTAL STATE CLASSIFIER

(71) Applicants: THE UAB RESEARCH FOUNDATION, BIRMINGHAM, AL (US); The Board of Regents of the University of Texas System, Austin, TX (US)

(72) Inventors: Adeel Ilyas, Birmingham, AL (US); Sandipan Bankim Behari Pati, Missouri City, TX (US)

(21) Appl. No.: 18/430,270

(22) Filed: Feb. 1, 2024

Related U.S. Application Data
(60) Provisional application No. 63/482,898, filed on Feb. 2, 2023.

(51) Int. Cl. A61B 5/00; A61B 5/293; A61B 5/374; G16H 10/60; G16H 50/70

(52) U.S. Cl. CPC A61B 5/7275; A61B 5/374; A61B 5/4094; A61B 5/7257; A61B 5/7267; A61B 5/746; G16H 10/60; G16H 50/70; A61B 5/293

(57) ABSTRACT

The present disclosure describes various embodiments of systems, apparatuses, and methods for predicting an onset of a seizure by identifying a pro-octal state in advance of the seizure, potentially hours prior to seizure onset. One such method comprise acquiring, by a computing device, electroencephalography (EEG)-based features from brain activity electrical recordings of an individual; inputting, by the computing device, the EEG-based features into a deep neural network-based classifier; classifying, by the computing device using the deep neural network-based classifier, the EEG-based features to a real-valued principal dimension; and/or based on a value of the real-valued principal dimension, generating, by the computing device, a prediction of a seizure onset pro-ictal event.

### CROSS-REFERENCE TO RELATED APPLICATION

This application claims priority to co-pending U.S. provisional application entitled, “Pro-Ictal State Classifier,” having application No. 63/482,898, filed Feb. 2, 2023, which is entirely incorporated herein by reference.

### BACKGROUND

In the greater-than-50 million individuals with epilepsy worldwide, a dynamical brain disorder renders them susceptible to seizures that cause physical harm, diminish quality of life, and increase mortality risk. In these individuals, the random occurrence of seizures (sometimes referred to as “a bolt from the blue”) constitutes substantial disability.

Traditionally, neural activities in epilepsy are classified into inter-ictal, ictal, and post-ictal states. However, studies of continuous electroencephalography (EEG) have partially deconvoluted the canonical stochasticity of seizures into periods of heightened risk, thereby subdividing the inter-ictal state into pre- and pro-ictal states. Whereas pre-ictal states invariably herald seizure onset within seconds or minutes, their pro-ictal counterparts represent fluctuations in neural activity across longer time scales during which a propensity for seizures exists. Studies of continuous electroencephalography (EEG) suggest that seizures preferentially occur during periods of heightened risk typified by pathologic brain activities, termed pro-ictal states. However, the presence of (pathologic) pro-ictal states amongst a plethora of otherwise physiologic (e.g. sleep-wake cycle) states has not yet been established. Furthermore, the neural underpinnings of these putative pro-ictal states present a critical knowledge gap in implementing effective seizure-preventative therapies.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a study environment in accordance with embodiments of the present disclosure.

FIG. 2A shows a table of electroencephalography (EEG)-based features that are input into a deep neural network-based classifier in accordance with embodiments of the present disclosure.

FIGS. 2B-2C show a table of demographic and seizure characteristics of patients included in a cohort study of the present disclosure.

FIG. 3 shows violin plots of classifier-based principal dimension reduction of pro- and inter-ictal EEG at part A and receiver-operating-characteristic curves depicting the performance of the classifier in distinguishing pro-versus inter-ictal states at part B for the cohort study of the present disclosure.

FIG. 4 shows a plot of classifier output (Π) of the entirety of EEG obtained during inpatient evaluation for each participant in the cohort study of the present disclosure.

FIG. 5 shows a bar plot of the aggregate Shapley values of EEG-based features (power, nonlinear synchrony [H²], Lempel-Ziv complexity [LZC], weighted phase lag index [wPLI], and phase-amplitude coupling [PAC]) inputted into the classifier across all patients during distinct pro-ictal periods during the cohort study of the present disclosure.

FIG. 6 shows a schematic block diagram of a computing device that can be used to implement various embodiments of the present disclosure.

### DETAILED DESCRIPTION

The present disclosure describes various embodiments of systems, apparatuses, and methods for predicting an onset of a seizure by identifying a pro-octal state in advance of the seizure, potentially hours prior to seizure onset.

Briefly described, one embodiment of the method, among others, includes acquiring, by a computing device, electroencephalography (EEG)-based features and inputting the EEG-based features into a deep neural network-based classifier. The neural network-based classifier is configured to classify these EEG-based features to a real-valued principal dimension, Π, or score. Then, based on a value of score, a prediction of a seizure onset pro-ictal event can be generated by identifying a pro-ictal state. For example, in various embodiments, a pro-ictal state can be predicted for scores at or near 100, where inter-ictal states are assigned scores at or near 0.

To develop an embodiment of systems and methods of the present disclosure, a study was performed on a prospective, consecutive series of 15 patients with temporal lobe epilepsy (TLE) who underwent limbic thalamic recordings in addition to routine (cortical) intracranial EEG for seizure localization. Pro-ictal (45 minutes prior to seizure onset) and inter-ictal (4 hours removed from all seizures) EEG was divided into 10-minute, non-overlapping windows, which were randomly distributed into training and validation cohorts in a 1:1 ratio. A deep neural classifier was applied to distinguish pro- from inter-ictal brain activities.

In the first human study of continuous thalamocortical EEG, compelling evidence was observed that pro-ictal states exist in TLE and can be detected more than one half-hour prior to seizure onset. Temporal lobe epilepsy is the archetypical drug-resistant focal epilepsy often targeted for interventions including microsurgery, laser ablation, and neuromodulation. Unfortunately, fewer than 50% of patients achieve durable seizure freedom after surgical intervention. As we increasingly understand epilepsy to be a network-rather than focal-disease, emerging evidence suggests that aberrant network reorganization of temporal and extratemporal structures, including the limbic thalamus, underlies ictogenesis. The limbic thalamus modulates large-scale temporal networks, regulates states of vigilance, and is a potentially efficacious neuromodulatory target for temporal lobe epilepsy.

To date, EEG-based seizure risk paradigms are corticocentric, meaning that they are derived from solely cortical, and attempt to predict seizures minutes prior to onset. However, the present disclosure hypothesizes the existence of pro-ictal states (i.e. pathologic brain activities during periods of heightened seizure risk) many minutes—and potentially hours—prior to seizure onset. Furthermore, given the substantive roles of the limbic thalamus, the present disclosure aims to both reliably detect these pro-ictal states using thalamocortical EEG and also identify their EEG-based signatures.

A cohort study was approved by the institutional review board at the University of Alabama at Birmingham (IRB-170323005). Adults with drug-resistant, suspected temporal lobe epilepsy who required presurgical stereo-EEG (SEEG) evaluation based upon the recommendation of a multidisciplinary epilepsy conference were approached for study inclusion. In routine care, electrodes are strategically implanted in specific cortical structures to elucidate the epileptogenic zone. Generally, electrodes are not implanted into the thalamus as part of routine care. In this study, an electrode was implanted into the limbic thalamus by modifying the trajectory of one of the electrodes sampling frontal operculum or insula, obviating the need for an additional electrode and thus minimizing complications. The locations of thalamic and other contacts were confirmed by co-registering the post-implantation computed tomography with the preimplantation T1-weighted magnetic resonance imaging using affine transformations and verifying electrode entry within the target subnucleus. The patient selection process for thalamic implantation, consenting process, implantation technique, and complication profile have been previously reported.

Written informed consent was obtained from each patient who underwent thalamic electrode implantation and EEG recording for research purposes. Patients were included in whom 1) the seizure onset zone localized to the temporal lobe and 2) the thalamic electrode was confirmed to be within the limbic thalamus ipsilateral to the seizure onset zone.

Consistent with routine care, all participants underwent inpatient, continuous video-SEEG monitoring to capture ictal activities. The duration of monitoring was at the discretion of the attending epileptologist who was not necessarily a study investigator. During monitoring, EEG was annotated to mark the onset of seizures as well as periods of wakefulness and sleep. Following inpatient monitoring, anatamo-electro-clinical findings were reviewed to localize the seizure onset zone by multidisciplinary conference consensus, the majority of whom were not study investigators. For each patient, representative channels within the seizure onset zone and limbic thalamus were selected for analysis. The location from which seizures consistently originated was selected as the representative seizure onset zone channel.

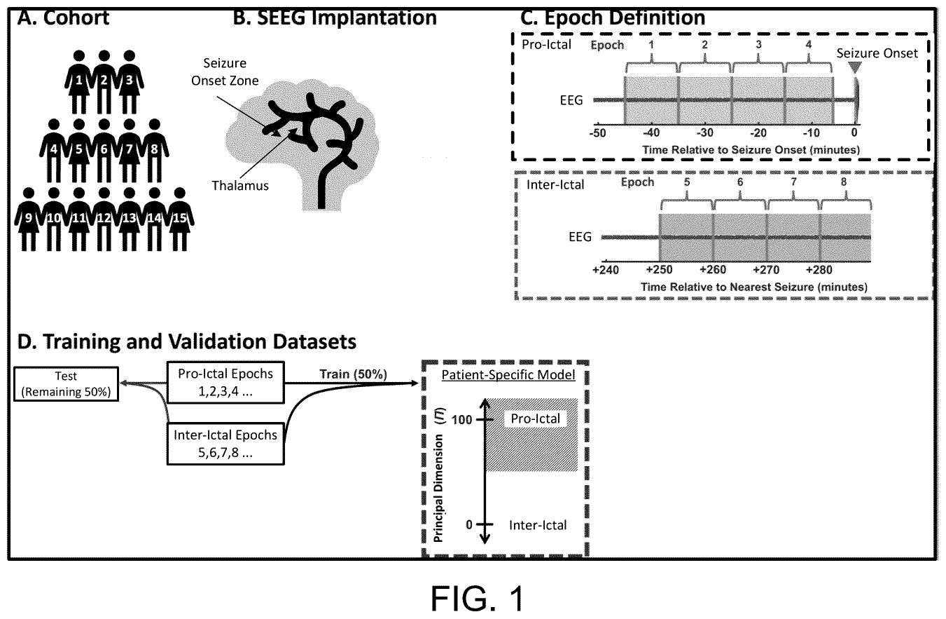

From the entirety of the inpatient monitoring period, inter-ictal epochs, defined as all timepoints 4 hours removed from the nearest seizure, were extracted. In addition, pro-ictal epochs beginning 5 minutes prior to seizure onset and extending 40 minutes retrograde were extracted. This duration of 40 minutes was derived from a Bayesian sample size calculation that was 90% powered to detect a difference between pro- and inter-ictal states. Epochs were divided into 10-minute, non-overlapping windows, which were randomly distributed into training and validation cohorts in a 1:1 ratio, as represented in the study environment of FIG. 1.

For the study environment, patients with medically refractory temporal lobe epilepsy undergoing stereo-electroencephalography (EEG) for pre-surgical workup of epilepsy were included as a cohort group (N=15), as represented in part A of FIG. 1. Each participant provided informed consent for electrode implantation into the thalamus for research purposes. EEG from the seizure onset zone and thalamus was captured during the inpatient monitoring period, as represented in part B of FIG. 1. The EEG was divided into 1) pro-ictal epochs, beginning 5 minutes prior to seizure onset and extending 40 minutes retrograde and 2) inter-ictal epochs, defined as all timepoints 4 hours removed from the nearest seizure, as illustrated in part C of FIG. 1. Epochs further were divided into 10-minute, non-overlapping windows, which are illustrated as numbered (i.e. 1 through 8), bracketed EEG, as depicted in part D of FIG. 1. These epochs were randomly distributed into training and validation cohorts in a 1:1 ratio. For each participant, a classifier used the training data to reduce EEG-based measures to a real-valued principal dimension, Π, in which pro- and inter-ictal states were assigned scores near 100 and 0, respectively. The training data were used to optimize the classifier's parameters. Subsequently, the optimized classifier's performance was assessed using the held-out, validation data. Both training and testing were performed in a patient-specific fashion.

Seizure onset zone and thalamic EEG were sampled at 2048 Hz (Natus Quantum, Natus Medical Incorporated, Pleasanton, CA). Missing EEG data from occasional hardware failure or device disconnection was omitted from the analysis (in total, 166 hours were missing from 1,618 hours of available data). In addition, EEG was manually evaluated for artifact contamination, and contaminated periods were also omitted. EEG was used to compute power, entropy, and synchrony (including weighted phase lag index and phase-amplitude coupling between the seizure onset zone and thalamus) measures, which were inputted into a deep neural network classifier. As discussed previously, the classifier reduced the EEG-based measures to a real-valued principal dimension, Π, in which pro- and inter-ictal states were assigned scores near 100 and 0, respectively. The training data were used to optimize the classifier's parameters. Subsequently, the optimized classifier's performance was assessed using the held-out, validation data.

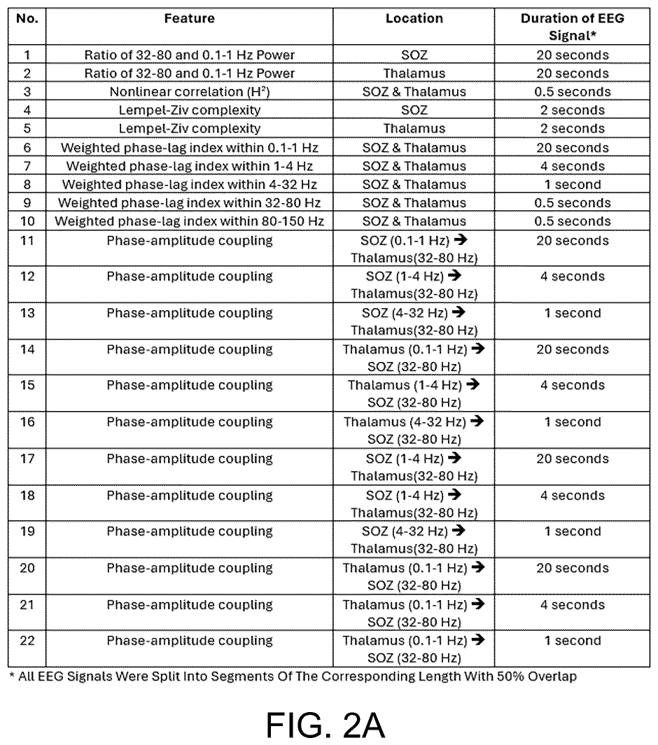

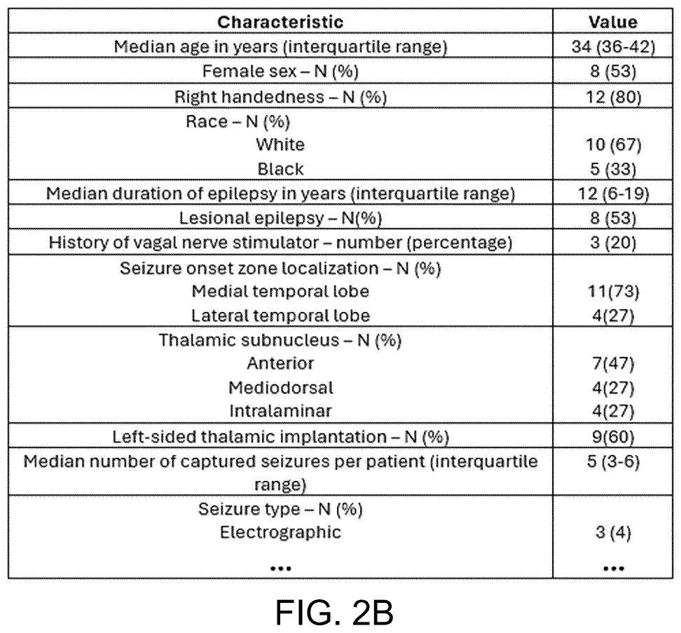

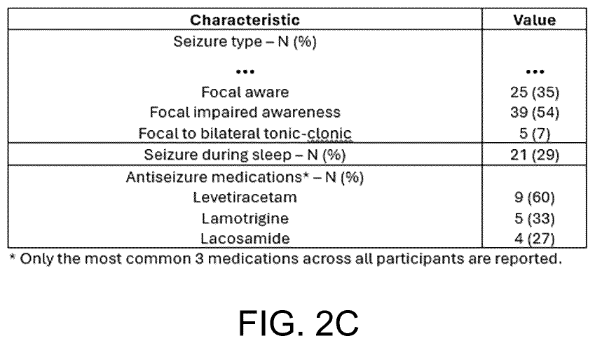

In particular, electroencephalography (EEG)-based features were inputted into a deep neural network-based classifier. The features are presented in the table of FIG. 2A. Prior to feature computation, the EEG was filtered to remove line noise and its harmonics using a zero-phase notch filter (third-order Butterworth) with 2 Hz width. To mitigate the filter-introduced ripples from EEG sharp transients (e.g. interictal discharges), the duration of EEG segments used in the analyzes was at least 500 milliseconds (and up to 20 seconds in some cases). Furthermore, the features were averaged across 2.5 seconds before being inputted into the neural network.

In an exemplary non-limiting implementation, power in the 32 to 80 Hz and 0.1 to 8 Hz frequency bands was obtained by applying the fast Fourier transform to the EEG segment (of 20 seconds) and summing the power within the respective frequency bands. The ratio of the power in these frequency bands was computed to yield Features 1 and 2.

Nonlinear correlation (i.e. h²) was obtained between the seizure onset zone (SOZ) and thalamus EEG segments (each of 0.5 second duration) by creating a scatterplot of amplitude (SOZ versus thalamus), dividing the scatter plot into 7 subsegments, and computing the linear regression curve for each subsegment independently. Thus, h² was computed as:

$${h}^{2} = \frac{{\sum }_{i} {y}_{i} - {\overline{y}}^{2} - {\sum }_{i} {y}_{i} - {f ( {x}_{i} )}^{2}}{{\sum }_{i} {y}_{i} - {\overline{y}}^{2}} ,$$

where i is the i^{th} sample across all 7 subsegments, yᵢ is the amplitude of the SOZ signal, y̅ is the segment-specific mean amplitude of the SOZ signal, and f(xᵢ) is the estimated amplitude of the SOZ signal (in each segment, estimated by linear regression). Feature 5 was the resulting h² value for the entire EEG segment.

The EEG segment (of 2 seconds) was binarized by computing the instantaneous amplitude via the Hilbert transform and using the median amplitude as the threshold. The binarized signal was compressed using the Lempel-Ziv 1978 (LZ78) compression algorithm, yielding a dictionary of unique, non-overlapping binary subsequences. The number of these subsequences was extracted to yield Features 3 and 4.

Weighted phase lag index (wPLI) was calculated between the SOZ and thalamus in different frequency bands: 0.1 to 1 Hz, 1 to 4 Hz, 4 to 32 Hz, 32 to 80 Hz, and 80 to 150 Hz. The instantaneous phase within the SOZ and thalamus EEG segments (each of 20, 4, 1, 0.5, and 0.5 seconds, respectively for the frequency bands of interest) was obtained via the Hilbert transform of the bandpass-filtered signal. The wPLI between the SOZ and thalamus was then computed within each frequency band as described by Vinck et al. The resulting wPLI values became Features 6 to 10.

Phase-amplitude coupling (PAC) was computed both within and between the SOZ and thalamus thereby resulting in 4 phase and amplitude combinations (i.e. SOZ phase modulating SOZ amplitude, SOZ phase modulating thalamus amplitude, thalamus phase modulating SOZ amplitude, and thalamus phase modulating thalamus amplitude). The low (i.e. phase) frequency bands were 0.1 to 1 Hz, 1 to 4 Hz, and 4 to 32 Hz. The high (i.e. amplitude) frequency band was 32 to 80 Hz (i.e. gamma). PAC of the SOZ and thalamus EEG segments (each of 20, 4, and 1 seconds, respectively for the different phase frequency bands) was computed on the bandpass-filtered signal as described by Canolty et al. The resulting PAC values became features 11 to 22.

The 22 features were computed from continuous-EEG 10-minute pro- and inter-ictal epochs and resampled to 500 milliseconds (the shortest duration across all features). These resampled features were then averaged using 2.5 second windows (thus each window contained 9 sets of 22 features) with 50% overlap, yielding 22 features for every 1.25 seconds of EEG. Thus, each 10-minute epoch contained 480 sets of the 22 features.

The pro- and inter-ictal 10-minute epochs were each randomly divided into training and validation sets in a 1:1 ratio. The training data was used to train a dense neural network consisting of 7 layers and 1 output layer, which outputted a real-valued number, Π. The objective function was to minimize the root-mean-squared error of Π−100 and Π−0 for the pro- and inter-ictal data points, respectively.

For statistical analysis purposes, statistical comparisons were performed using unpaired, two-tailed Bayes factor t-tests, and a stringent threshold of 100 (i.e. log₁₀BF>2) defined significance for the primary analysis. Unless otherwise indicated, all tests of significance were performed on the held-out validation data. Statistical tests were computed in R (R Project for Statistical Computing, version 3.6.3). The primary analysis was the classifier's ability to distinguish a pro-ictal state by statistically comparing Π from the pro-versus inter-ictal EEG segments. In addition, the classifier's area under the receiver-operating-characteristic curve (AUC) was computed. The significance of the AUC was expressed as a z-score over 100 simulations of training and evaluating the classifier on randomly shuffled pro- and inter-ictal EEG data, thus representing z-score above chance classification (i.e. AUC of 0.5).

Secondary analyses included the duration prior to seizure onset during which the pro-ictal state was statistically distinguished in each patient. During these statistically distinguished pro-ictal periods, the significance of each of the imputed features was expressed using Shapley values, which express the marginal contribution of each feature to the output (i.e. Π) weighted over all possible feature combinations.

The following results of the cohort study were observed. To begin, between September 2017 and 2021, 28 patients underwent thalamic implantation, of whom 15 met inclusion criteria and were included in the study. Reasons for exclusion of the 13 patients were seizure onset both within and outside the temporal lobe (i.e. temporal-plus epilepsy, N=9), seizure onset outside the temporal lobe (i.e. extra-temporal lobe epilepsy, N=3), and electrode localization outside the limbic thalamus (N=1). The seizure onset zone localized to mesial and lateral temporal structures in 11 (73%) and 4 (27%) patients, respectively. The seizure onset zone was left-sided in 9 (60%) patients. The thalamic electrode was placed within the anterior, mediodorsal, and intralaminar subnuclei of the limbic thalamus in 7 (47%), 4 (27%), and 4 patients, respectively. Across all patients, the total duration of video-SEEG recording was 77.4 days (median, 4.8 days per patient; interquartile range [IQR], 3.7 to 5.8 days), during which a total of 72 seizures (median, 5 seizures per patient; IQR, 3 to 6 seizures per patient) were captured and analyzed. Electrographic, focal aware, focal impaired awareness, and focal to bilateral tonic-clonic seizures constituted 4%, 35%, 54%, and 7% of all seizures, respectively. Seizures occurred during sleep (defined by clinical and electrographic criteria) in 29% of cases. Demographic and seizure characteristics of the included patients are presented in the table of FIGS. 2B-2C. Overall, study participants were representative of the typical population of patients with temporal lobe epilepsy. The participants in the present study demonstrated the expected ratio of male to female (53% were female). Median age of participants (34 years) was consistent with the average age of TLE diagnosis.

In each participant, the 40-minute pro-ictal epochs (each of which began 5 minutes prior to seizure onset) were converted into the principal dimension (Π) using the neural network classifier. In this case, both the training and validation pro-ictal data was combined to guarantee a contiguous 40-minute pro-ictal interval for analysis. It is apparent the probability that the participant-specific classifier distinguished a pro-ictal state within each participant by random chance is infinitesimally small as Bayes factors were greater than 1010 in each participant. Therefore, it is reasonably assumed that these classifiers represented a reliable means of distinguishing pro-versus inter-ictal states within each participant. Under this assumption, within-participant statistical analyses were performed to determine the duration of these pro-ictal states relative to seizure onset. The pro-ictal epoch Π-values across all included seizures were statistically compared to that of inter-ictal Π-values (from both training and validation datasets) with a two-tailed Bayes factor t-test, applied at each timepoint. In this manner, timepoints that contained statistically distinct pro-ictal states were obtained in each participant with the available seizure data. Statistically distinct pro-ictal states were detected in each participant. Across all patients, taking 500 random 2.5-second samples from each of pro- and inter-ictal EEG, mean Π was 86 and 19 for pro- and inter-ictal states, respectively (log₁₀BF>10), as shown in part A of FIG. 3. The median AUC of the classifier was 0.92 (IQR, 0.90 to 0.96), which corresponded to a median 235 (IQR, 181 to 305) z-scores above chance, as shown in part B of FIG. 3.

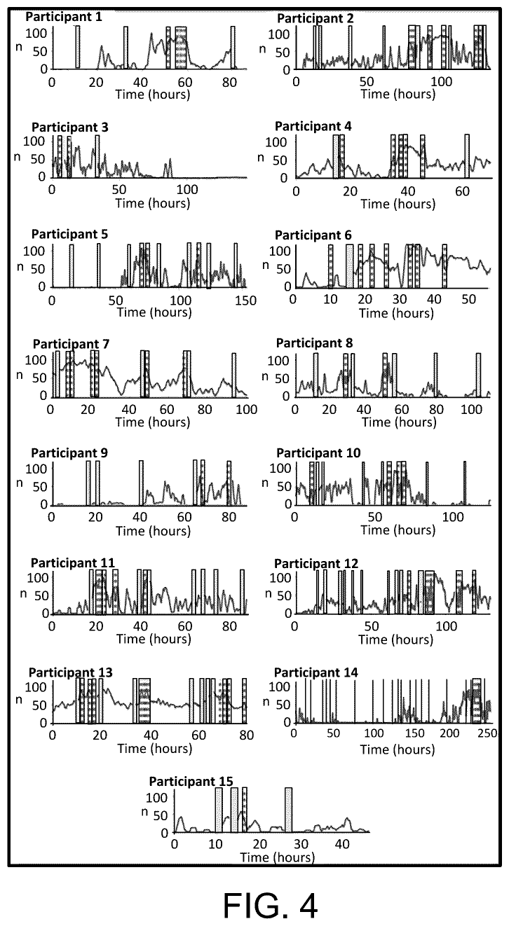

During inpatient monitoring, Π appeared to correlate with seizure activity in each patient. As such, Π increased in the pre-ictal period and decreased at timepoints at least 4 hours removed from seizure activities, as shown in FIG. 4. Furthermore, pro-ictal states were statistically distinguished at least 45 minutes prior to seizure onset in all but 2 patients (participants 3 and 8), in whom they were distinguished up to 35 minutes prior. During these statistically defined pro-ictal periods, the most important contributing features were power and phase-amplitude coupling (PAC), as shown in FIG. 5.

Changes in power and phase-amplitude coupling during pro-versus inter-ictal states were explored. For example, in each participant, thalamic electroencephalography (EEG) signals during statistically distinguished pro-ictal periods was extracted and binned into nonoverlapping windows of 20-seconds duration. Similarly, thalamic EEG during the inter-ictal period was binned into nonoverlapping windows of 20-seconds duration. The number of inter-ictal bins far exceeded the number of pro-ictal bins; therefore, the two sets were equated by randomly selecting an appropriate number of bins from the inter-ictal set. The fast Fourier transform was applied to the 20-second EEG segment within each bin, yielding the corresponding periodogram for that bin. The mean periodogram within the pro- and inter-ictal cohorts was computed by averaging power across all bins within each frequency band. The mean periodogram of the inter-ictal cohort was subtracted from that of the pro-ictal cohort to determine a power-based signature of the pro-ictal state. This signature was expressed as a z-score by bootstrapping (subtracting inter-ictal from pro-ictal periodograms after randomly shuffled pro- and inter-ictal bins 1000 times). The significance of each frequency band was computed by comparing the power in that band in the pro- and inter-ictal cohorts using two-tailed Bayes factor t-tests. Thus, distinct changes in power within the thalamus were observed in 7 of 15 (47%) participants, of whom 5 demonstrated increased power below 30 Hz.

In 7 of the remaining 8 patients, although changes in power were observed at low frequencies, these changes did not meet the statistical threshold. For example, in each patient, a synchrony-based signature of statistically distinguished pro-ictal periods was computed by first binning pro- and inter-ictal electroencephalography (EEG) signals into 20-second windows. Next, thalamic EEG was bandpass filtered at low frequencies (0.1 to 36 Hz in 2-Hz steps) and seizure onset zone (SOZ) EEG, at high frequencies (36 to 300 Hz in 2-Hz steps). Phase amplitude coupling in each 20-second window was computed between the thalamus (providing phase) and SOZ as described by Tort et al. The signature (difference between pre- and inter-ictal phase amplitude coupling) was expressed as a z-score corresponding to each low (i.e. phase) and high (i.e. amplitude) frequency band pair using bootstrapping techniques. The significance of each z-score was determined using two-tailed Bayes factor t-tests. By applying the aforementioned technique, distinct pro-ictal signatures (thalamocortical phase amplitude coupling signatures of pro-ictal states) that were distinguished from physiologic inter-ictal activities were detected in 13 of 15 participants. In some participants, these signatures were marked by increased thalamocortical synchrony while in others, decreased synchrony was observed. These findings may be related to precise networks that were sampled in each participants, which were non-standardized across participants.

In the cohort study of thalamic SEEG limited by a small sample size of 15 patients, evidence was provided that pro-ictal states could be identified in the majority, but not all patients that were studied with temporal lobe epilepsy. Pro-ictal states were detected at horizons of at least one half-hour prior to seizure onset. Continuous EEG spanning multiple days of inpatient seizure monitoring was analyzed, where half of the EEG data was randomly selected for classifier training and the remaining half for testing. Furthermore, both training and testing samples included periods of physiologic (e.g. wakefulness and sleep) as well as pathologic (e.g. seizures) states. As such, the classifier is unlikely to have simply detected specific states of vigilance per se, given the extended periods (more than 50 hours in most patients) of low classifier-based pro-ictal scores (i.e. Π<50) during which sleep-wake cycles would have occurred.

In accordance with the present disclosure, a novel yet pragmatic approach is applied to pro-ictal state detection, utilizing thalamic EEG. The limbic thalamus exerts a diverse influence on cortical activity and modulates arousal, awareness, attention, and sensorimotor processing. Furthermore, a mechanistic role of the limbic thalamus in focal epilepsies, first proposed by Penfield and Jasper in the mid-1900s, is becoming increasingly established. In addition, thalamic neuromodulation appears to be an effective therapy for select patients with temporal lobe epilepsy. Therefore, the present disclosure rationalized (and observed) that thalamocortical dynamics readily distinguish physiologic and pathologic brain states in temporal lobe epilepsy thereby providing a means to detect pro-ictal states.

The goal of seizure prediction is to develop adaptive therapies that adjust treatment according to seizure risk. Currently, efforts are underway to develop effective EEG-based adaptive neuromodulation technologies that apply intracranial stimulation during periods of heightened seizure risk. A pragmatic means of EEG-based pro-ictal state detection is crucial to the success of adaptive neuromodulation. Therefore, in various embodiments, EEG recordings are utilized from only, but not limited to only being utilized from, two electrode contacts (i.e. one in the seizure onset zone and one in the thalamus), a capability present in contemporary neuromodulation technologies. For example, certain approaches of the present disclosure may be simplified to a single electrode. Detecting pro-ictal states from thalamic EEG has profound implications for neuromodulation therapy in certain generalized epilepsies in which diffuse brain regions, including the thalamus, are instantaneously recruited. Conversely, utilizing EEG from all sampled brain regions, may also improve pro-ictal state detection accuracy, but may not be practical for certain applications.

Mechanistic insights into the neural substrates that underly periods of heightened seizure risk are lacking but much needed. As a secondary aim, the present disclosure attempts to elucidate specific EEG-based features that most contribute to successful pro-ictal state classification (during epochs 5 to 45 minutes prior to seizure onset that were statistically distinct from inter-ictal states). Based on this analysis, participant-specific changes in thalamic power and thalamocortical synchrony were observed, the latter changes being more robust across participants. Interestingly, phase-amplitude coupling changes within the seizure onset zone that correspond to the pre-ictal state (approximately one-half minute prior to seizure onset) have been observed in human focal epilepsy. It was observed that changes in synchrony during pro-ictal states are distinct from physiologic changes during wakefulness and sleep. Thus, it is speculated that modulation of thalamocortical synchrony during pro-ictal periods may be an efficacious therapeutic approach to the treatment of temporal lobe epilepsy in patients undergoing neuromodulation therapies although this was not tested in our study.

Interictal epileptiform discharges are paroxysmal, brief depolarization shifts that manifest in stereo-EEG as spikes (20 to 70 millisecond duration) or sharp waves (70 to 200 millisecond duration). Although these events are sporadic in most patients with epilepsy, their event rates are known to change before or after a seizure. However, it is unlikely that interictal epileptiform discharges meaningfully influenced the individualized classifiers used in the aforementioned study. The time windows (ranging from 500 milliseconds to 20 seconds) of the inputted features were much too long to detect transient depolarizations. Furthermore, interictal epileptiform discharges are known to increase during certain sleep stages in patients with focal onset epilepsies. Conversely, Π was observed to not significantly change during physiologic wakefulness versus sleep.

Based on the present cohort study, the influence of number of captured seizures on the results was evaluated. Across all participants, using AUC as a surrogate for classifier performance, the correlation of number of captured seizures with z-scores above chance classification was computed and was observed to have a correlation coefficient of 0.21, indicating that the number of seizures did not influence classifier performance. As expected, the two participants with the least amount of available training data, participants 6 and 13, also had the worst classifier performance.

While the present study is limited by a small sample of size of 15 patients and heterogeneity of demographic factors, epilepsy characteristics (e.g. lesions versus non-lesional), medication adjustments, and duration of continuous EEG recording in the patients, the present disclosure shows that pro-ictal states exist in temporal lobe epilepsy using individualized EEG-based signatures. To facilitate the capture of seizure events during inpatient monitoring, antiseizure medications were withdrawn at the discretion of the attending epileptologist, which inherently increases seizure risk. Serum levels of these antiseizure medications were not measured during periods of weaning and thus we cannot conclusively compute the extent to which pro-ictal state scores (Π) were independent of antiseizure medications changes. Nonetheless, increases in Π were observed during the first 24 hours of admission when antiseizure medications were either maintained at outpatient levels or had just begun to be weaned. Patients were not kept inpatient beyond the time necessary to complete their clinical evaluations. Thus, an equal amount of inter-ictal and pro-ictal data was not obtained across all participants. However, this was mitigated by randomly splitting the data into training and validation cohorts in the same ratio in each participant. While the associated findings are limited to temporal lobe epilepsy, similar methodology could possibly be applied to detect pro-ictal states in other epilepsies. Furthermore, our data are subject to referral and treatment biases associated with a single-center study in a Level IV epilepsy center.

In brief, based upon thalamocortical EEG, pro-ictal states (pathologic brain activities during periods of heightened seizure risk) could be identified in most patients with temporal lobe epilepsy and were detected in our small sample, more than one half-hour prior to seizure onset. The existence of non-physiologic brain states during periods of heightened seizure risk implies that adaptive neuromodulation therapies may be delivered at clinically meaningful horizons to potentially arrest ictogenesis.

As described herein, a seizure prediction system refers to a system that applies a prediction algorithm to data in order to generate a prediction model for making predictions on the onset of a seizure event. A prediction system can be a prediction server that applies the prediction algorithm and performs the related methods, wherein the prediction server comprises a digital processing device comprising at least one processor, an operating system configured to perform executable instructions, a memory, and a computer program including instructions executable by the digital processing device to create a server application.

As used herein, “risk” refers to the likelihood of occurrence of an event, such as a seizure. A “risk prediction” refers to the likelihood of occurrence of an event, such as a seizure. A risk prediction is calculated using a prediction model generated by a prediction algorithm. The prediction algorithm may also comprise machine learning methods in generating the prediction model. A prediction model can be a formula comprising parameters that determine the likelihood of a seizure. For example, a prediction model can be a multiple linear regression model or formula, a classifier, and/or trained algorithm generated by the application of a machine learning algorithm to a dataset comprising EEG or other types of brain recordings.

In various embodiments, a prediction system involves a prediction server that executes a software application having a framework for a seizure prediction model. The software application outputs a prediction of a risk associated with onset of a seizure by an individual. In general, the software application uses brain activity of the individual to compute a risk score with values that vary between 100 (high) and 0 (low).

The software application may be in any computer programming languages such as Perl, PHP, Python, Ruby, JavaScript (Node), Scala, Java, Go, ASP.NET, ColdFusion, etc. In some embodiments, the software application may use machine learning principles including Support Vector Machine (SVM), Random Forest (RF), Naive Bayes Classifier, neural networks, deep neural networks, logistic regression, etc., for classification. A prediction algorithm may comprise generating a seizure risk model using machine learning on recordings of brain activities, wherein the machine learning is selected from Support Vector Machine (SVM), Random Forest (RF), Naive Bayes Classifier, neural networks, deep neural networks, and logistic regression.

In various embodiments, the software modules disclosed herein comprise a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application.

Next, FIG. 6 depicts a schematic block diagram of a computing device 600 that can be used to implement various embodiments of the present disclosure, such as a prediction server. An exemplary computing device 600 includes at least one processor circuit, for example, having a processor (CPU) 602 and a memory 604, both of which are coupled to a local interface 606, and one or more input and output (I/O) devices 608. The local interface 606 may comprise, for example, a data bus with an accompanying address/control bus or other bus structure as can be appreciated. The computing device 600 further includes Graphical Processing Unit(s) (GPU) 610 that are coupled to the local interface 606 and may utilize memory 604 and/or may have its own dedicated memory. The CPU and/or GPU(s) can perform various operations such as image enhancement, graphics rendering, image/video processing, recognition (e.g., text recognition, object recognition, feature recognition, etc.), image stabilization, machine learning, filtering, image classification, and any of the various operations described herein.

Stored in the memory 604 are both data and several components that are executable by the processor 602. In particular, stored in the memory 604 and executable by the processor 602 are code for implementing one or more neural networks 611 (e.g., artificial and/or convolutional neural network models) and a software application 670 (e.g., using the neural network models 611) for building seizure risk model(s) and predicting an onset of a seizure event. Also stored in the memory 604 may be a data store 614 and other data. The data store 614 can include an electronic repository or database relevant recordings of brain activities. In addition, an operating system may be stored in the memory 604 and executable by the processor 602. The I/O devices 608 may include input devices, for example but not limited to, a keyboard, mouse, EEG monitoring devices, etc. Furthermore, the I/O devices 608 may also include output devices, for example but not limited to, a printer, display, etc.

Certain embodiments of the present disclosure can be implemented in hardware, software, firmware, or a combination thereof. If implemented in software, the logic or functionality for building seizure risk model(s) and predicting a seizure onset pro-ictal event is implemented in software or firmware that is stored in a memory and that is executed by a suitable instruction execution system. If implemented in hardware, such logic or functionality can be implemented with any or a combination of the following technologies, which are all well known in the art: discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Thus, one or more or more of the components described herein that includes software or program instructions can be embodied in any non-transitory computer-readable medium for use by or in connection with an instruction execution system such as a processor in a computer system or other system. The computer-readable medium can contain, store, or maintain the software or program instructions for use by or in connection with the instruction execution system.

The computer-readable medium can include physical media, such as, magnetic, optical, semiconductor, or other suitable media. Examples of a suitable computer-readable media include, but are not limited to, solid-state drives, magnetic drives, flash memory. Further, any logic or component described herein can be implemented and structured in a variety of ways. One or more components described can be implemented as modules or components of a single application. Further, one or more components described herein can be executed in one computing device or by using multiple computing devices.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. A method comprising:
acquiring, by a computing device, electroencephalography (EEG)-based features from brain activity electrical recordings of an individual;
inputting, by the computing device, the EEG-based features into a deep neural network-based classifier;
classifying, by the computing device using the deep neural network-based classifier, the EEG-based features to a real-valued principal dimension; and
based on a value of the real-valued principal dimension, generating, by the computing device, a prediction of a seizure onset pro-ictal event.

2. The method of claim 1, further comprising alerting the individual of the prediction of the seizure onset pro-ictal event.

3. The method of claim 1, wherein the seizure onset pro-ictal event is predicted to occur at least 30 minutes before the individual experiences a seizure.

4. The method of claim 1, wherein the brain activity electrical recordings comprise continuous thalamocortical EEG electrical recordings.

5. The method of claim 1, wherein the EEG-based features are classified based on an EEG-based signature.

6. The method of claim 5, wherein the EEG-based signature comprises a power-based signature of the seizure onset pro-ictal event.

7. The method of claim 1, further comprising acquiring the brain activity electrical recordings from two electrode contacts with one coupled to a seizure onset zone of the brain of an individual and another coupled to a thalamus structure of the brain of the individual.

8. The method of claim 1, wherein the EEG-based features comprise power and/or phase-amplitude coupling features between a seizure onset zone and thalamus of a brain of an individual.

9. A system comprising:
at least one processor; and
memory configured to communicate with the at least one processor, wherein the memory stores instructions that, in response to execution by the at least one processor, cause the at least one processor to perform operations comprising:
acquiring, by a computing device, electroencephalography (EEG)-based features from brain activity electrical recordings of an individual;
inputting, by the computing device, the EEG-based features into a deep neural network-based classifier;
classifying, by the computing device using the deep neural network-based classifier, the EEG-based features to a real-valued principal dimension; and
based on a value of the real-valued principal dimension, generating, by the computing device, a prediction of a seizure onset pro-ictal event.

10. The system of claim 9, wherein the operations further comprise alerting the individual of the prediction of the seizure onset pro-ictal event.

11. The system of claim 9, wherein the seizure onset pro-ictal event is predicted to occur at least 30 minutes before the individual experiences a seizure.

12. The system of claim 9, wherein the brain activity electrical recordings comprise continuous thalamocortical EEG electrical recordings.

13. The system of claim 9, wherein the EEG-based features are classified based on an EEG-based signature.

14. The system of claim 13, wherein the EEG-based signature comprises a power-based signature of the seizure onset pro-ictal event.

15. The system of claim 9, wherein the operations further comprise acquiring the brain activity electrical recordings from two electrode contacts with one coupled to a seizure onset zone of the brain of an individual and another coupled to a thalamus structure of the brain of the individual.

16. The system of claim 9, wherein the EEG-based features comprise power and/or phase-amplitude coupling features between a seizure onset zone and thalamus of a brain of an individual.

17. A non-transitory computer readable medium comprising machine readable instructions that, when executed by a processor of a computing device, cause the computing device to at least:
acquire electroencephalography (EEG)-based features from brain activity electrical recordings of an individual;
input the EEG-based features into a deep neural network-based classifier;
classify, using the deep neural network-based classifier, the EEG-based features to a real-valued principal dimension; and
based on a value of the real-valued principal dimension, generate a prediction of a seizure onset pro-ictal event.

18. The non-transitory computer readable medium of claim 17, wherein the instructions further cause the computing device to alert the individual of the prediction of the seizure onset pro-ictal event.

19. The non-transitory computer readable medium of claim 17, wherein the seizure onset pro-ictal event is predicted to occur at least 30 minutes before the individual experiences a seizure.

20. The non-transitory computer readable medium of claim 17, wherein the brain activity electrical recordings comprise continuous thalamocortical EEG electrical recordings.
